(19) United States
(12) Patent Application Publication
(10) Pub. No.: US 2019/0307627 A1
(43) Pub. Date: Oct. 10, 2019

(54) Device to Provide Optimal Positioning for Endotracheal Intubation or Cricothyroidotomy in the Emergency Department, in the Operating Room, and by First Responders on the Scene of Emergency Situations

(71) Applicant: Eric S. Moon, D. O., Cedar Lake, IN (US)

(72) Inventor: Eric S. Moon, D. O., Cedar Lake, IN (US)

(21) Appl. No.: 16/374,972

(22) Filed: Apr. 4, 2019

Related U.S. Application Data
(60) Provisional application No. 62/652,569, filed on Apr. 4, 2018.

(51) Int. Cl. A61G 13/12; A61M 16/04

(52) U.S. Cl. CPC A61G 13/1215; A61G 13/126; A61M 16/0488; A61M 16/0472

(57) ABSTRACT

By providing elevation of the upper back above the hospital bed, extension of the entire cervical spine will provide better visualization than achieved by the prior art such as the sniffing position. An additional benefit of using this device is that with full extension of the neck, the position for cricothyroidotomy is also optimized, should standard endotracheal intubation fail. This device will be of particular benefit in the intubation of obese patients, providing good extension and adequate visualization without the need for excessive strength to manipulate the patient's head and neck. The same position also facilitates placement of central venous catheters in the internal jugular or subclavian veins in obese or kyphotic patients.

### RELATED APPLICATIONS

The present application claims priority to and all the benefits of U.S. Provisional Patent Application No. 62/652,569, filed on Apr. 4, 2018, which is hereby expressly incorporated herein by reference in its entirety.

### TECHNICAL FIELD

This new device uses or creates a new position to facilitate endotracheal intubation, cricothyroidotomy, and other procedures. It is derived from a new model of airway visualization based on a better use of manipulated position. It is simple to use, portable and lightweight.

### BACKGROUND

### I. ENDOTRACHEAL INTUBATION

Endotracheal intubation is the placement of a plastic tube through the mouth, past the vocal cords, and into the trachea where it functions to provide oxygen and protect against aspiration in critically ill patients. It is the most vital, life-saving skill that an emergency physician must have. An example of endotracheal intubation is shown in FIGS. 1 and 2.

### II. AIRWAY ANATOMY

The path from the mouth to the trachea is nonlinear. There are several obstacles that the endotracheal (ET) tube must pass, including the tongue, posterior pharynx, and epiglottis. Proper alignment of the head and neck is necessary to provide a clear view of the vocal cords. Without proper alignment, the ET tube cannot get past the structures which stand in the way. Without a good view of the vocal cords, the ET tube is more likely to pass into the esophagus than into the trachea. Improper placement of the endotracheal tube will result in an inability to provide the patient with vital oxygen.

### III. CRICOTHYROIDOTOMY

If the physician fails to place an ET tube in the proper position, oxygen depletion will quickly result in brain damage, cardiac arrest, and death. These events will occur within minutes. In the most emergent scenarios, the window of opportunity may be measured in seconds. In such cases, there will not be time for a second attempt. Without securing the airway, the patient will quickly die.

In these critical situations, failed intubation is followed by cricothyroidotomy to secure the airway. As shown in FIGS. 3, 4 and particularly 5, the cricothyroidotomy procedure involves cutting the anterior neck to expose the trachea, puncturing the cricothyroid membrane, and inserting the endotracheal tube directly into the trachea at the site of puncture. By the time a cricothyroidotomy is being performed, the physician usually has only a matter of seconds before the patient dies or suffers permanent disabling damage to the brain. As with endotracheal intubation, proper positioning is essential to facilitate the procedure.

### IV. THE PRIOR ART “SNIFFING POSITION” FOR AIRWAY VISUALIZATION DURING ENDOTRACHEAL INTUBATION

Direct visualization of the cords was first achieved in 1895 by German Otolaryngologist, Dr. Alfred Kirstein, who invented the laryngoscope. In 1913 Dr. Chevalier Jackson described a technique in which an assistant elevated the patient's head to provide better visualization. As shown in FIG. 6, in 1936, anesthesiologist, Dr. Ivan Magill described this position as the “sniffing position.” It has since been accepted as the best method of visualizing the airway during endotracheal intubation. The sniffing position is described as forward flexion of the neck with backward extension of the head.

As shown in FIG. 7, in 1944 Dr. Freda Bannister and Dr. Ronald MacBeth critically evaluated the sniffing position and described the “Three Axis Alignment” theory. Their theory proposed that it was proper alignment of the oral, pharyngeal, and laryngeal axes that made the sniffing position ideal for visualization of the vocal cords during laryngoscopy.

For decades after its initial description, the sniffing position was taught dogmatically in anesthesia, ENT, surgical, and emergency medicine training. It was not critically analyzed until the turn of the century when Dr. Frederic Adnet began to question the three axis theory and whether or not the sniffing position was advantageous. Later, Dr. Keith Greenland redefined airway anatomy in terms of “two curves” rather than “three axes.” He used his new anatomic model to reconfirm the benefit of the sniffing position for endotracheal intubation.

Today, over a century after Kirstein invented the laryngoscope, there has been incredible progress in the technology used to visualize the vocal cords during endotracheal intubation. In spite of this, there has been no change in the understanding of the anatomy, and a better method of positioning has not been proposed.

As shown in FIG. 8, sowing a comparison of patients in neutral (FIG. 8a) and “ramped” (FIG. 8b) positions demonstrating flexion of the neck and extension of the head, as in the “sniffing” position. in 2000, Levitan and Ochroch described a variation of the sniffing position achieved by elevation of the shoulders in obese patients. This was later described by Collins et al as the “ramped position” but the underlying premise is the same: that flexion of the neck is essential for visualization of the vocal cords. Thus, the underlying mechanics of the original sniffing position have essentially gone unchallenged.

### V. OTHER PATENTED DEVICES FOR AIRWAY POSITIONING AND THEIR COMMON DIFFERENCE FROM THE PROPOSED DEVICE

Several devices attempting to optimize positioning of the airway are in the prior art. U.S. Pat. No. 7,127,758 B2 (Gabbay) issued Oct. 31, 2006 is an inflatable lift. U.S. Patent Publication No. 2001/0036354 A1 (Farnum) issued Feb. 17, 2011 is a mechanical crank. U.S. Pat. No. 6,751,818 B2 (Troop) issued Jun. 22, 2004 is a static, immobile wedge.

All of these devices are based upon the classic “sniffing” position or the more recent “ramped” position of airway visualization, with lower cervical flexion as a key factor in their design. In contrast, the device of the present invention is based upon an entirely new position that involves full extension of the entire cervical spine.

The device of the present invention also differs from previous devices in allowing the shoulders to drop backward for better exposure of the neck and chest, facilitating other invasive procedures. These procedures include cricothyroidotomy and placement of various central venous catheters.

By using radiographic imaging, it will be demonstrated that this new device will allow superior positioning and easier visualization of the vocal cords during endotracheal intubation. This difference will be particularly noticeable when intubating obese patients because it will obviate the need for operator strength to manipulate the patient's head and neck.

The device of the present invention is conceptually different and will prove to be operationally superior. Additional advantages and differences from the prior art will be apparent from the description and drawings.

### VI. BRIEF DESCRIPTION OF THE DRAWINGS

FIG. 1 is an elevated view of health care professionals intubating a patient;

FIG. 2 is a schematic sectional view of an intubation procedure similar to that shown in FIG. 1;

FIG. 3 is an elevated view of location points on a human body that are used for intubation procedures;

FIG. 4 is an elevated view of the cutting position for an intubation procedure on a patient;

FIG. 5 is a schematic view of four steps comprising parts of an intubation procedure;

FIG. 6 is an elevated schematic view of a prior art procedure;

FIG. 7 is an elevated schematic view of a prior art procedure;

FIG. 8a is an elevated schematic view of a first position for an obese patient;

FIG. 8b is an elevated schematic view of a second position for an obese patient;

FIG. 9 is an x-ray or radiographic view of a patient in a prior art position;

FIG. 10 is an x-ray or radiographic view of a patient in a neutral position;

FIG. 11 is an x-ray or radiographic view of a patient in a position similar to FIG. 9;

FIG. 12 is a schematic view of a prior art position;

FIG. 13 is a schematic view of a neutral position;

FIG. 14 is a schematic view of a position using the invention;

FIG. 15 is an elevated view of an obese patient in a neutral position;

FIG. 16 is an elevated view of an obese patient using the invention;

FIG. 17 is a top view of the positioning device;

FIG. 18 is an elevational side view of the positioning device;

FIG. 19 is a front elevational view of the positioning device; and

FIG. 20 is an elevated perspective view of the positioning device.

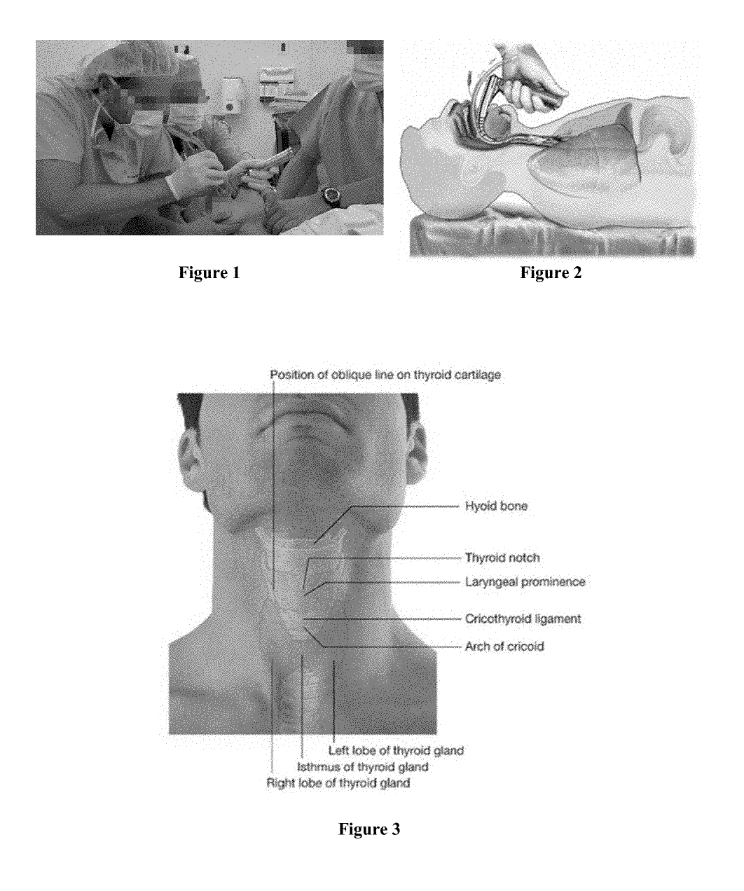

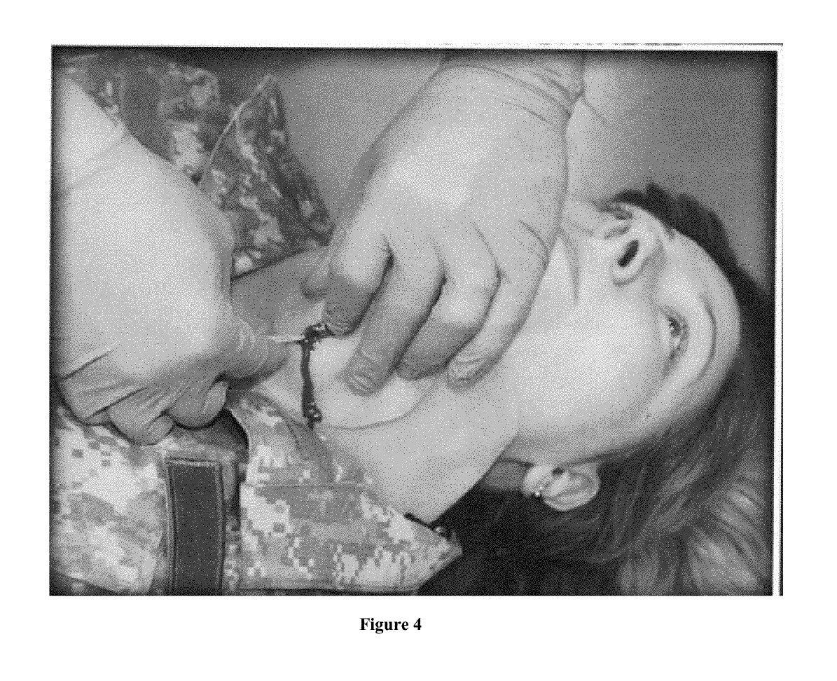

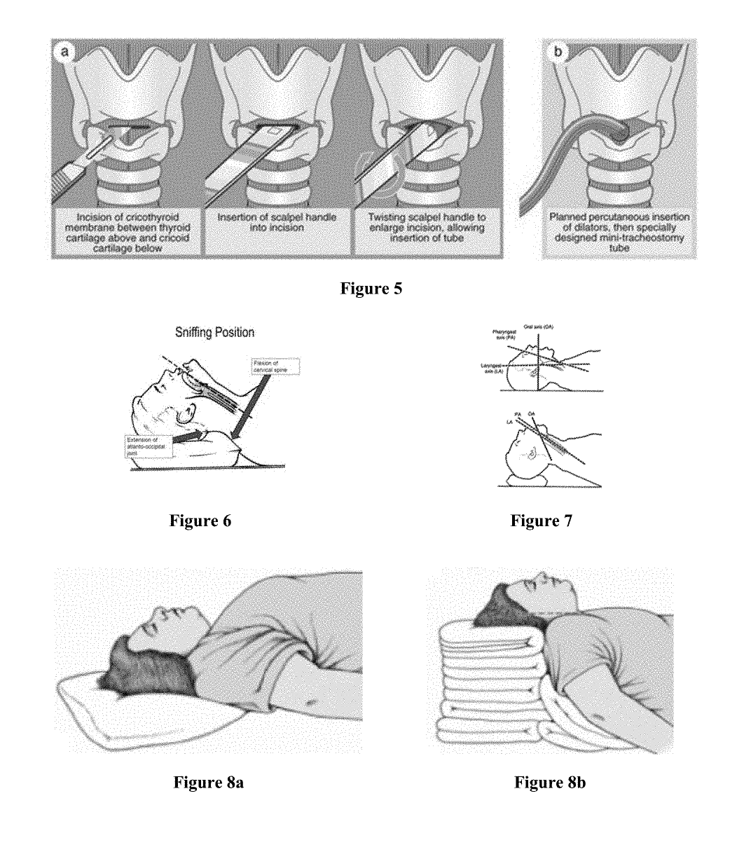

### VII. DETAILED DESCRIPTION OF A NOVEL DEVICE THAT USES A NEW POSITIONING MECHANISM INSTEAD OF THE TRADITIONAL “SNIFFING” AND “RAMPED” POSITIONS

The “sniffing” position and the newer variant known as the “ramped” position continue to be taught as the ideal methods of positioning for visualization of the airway during endotracheal intubation. Both positions require flexion of the neck as a key component. In the majority of patients, proper visualization is easily achieved by use of the laryngoscope alone, making debate over these positions irrelevant.

However, in a significant number of cases, certain individual characteristics of the patient make visualization difficult, complicating the process of intubation. Obesity in particular presents a great challenge, with difficulty greatly increasing with the size and weight of the patient. The sniffing and ramped positions have both been demonstrated by many to be beneficial for obese patients in particular.

In his 2001 critique of the sniffing position's validity, Adnet did MRI images of patients in three positions: neutral position, simple extension, and the sniffing position with the neck flexed and the head extended. He concluded that the sniffing position provided no added benefit over simple extension of the neck, as demonstrated below. However, what was not appreciated in these images was that simple “extension” of the neck inherently caused flexion of the lower neck. Similarly, “extension of the head” also induced extension of the upper neck.

Thus, in Adnet's study, the “sniffing position” and “simple neck extension” both caused flexion at C6 and C7 while the rest of the neck was extended backwards. These were essentially variations of the same position and a valid comparison to true neck extension was not actually achieved.

Further inspection of the anatomy demonstrates that the vocal cords are located at about the level of the 5th cervical vertebral body. Because of this, flexion at the base of the neck has little effect on the position of the vocal cords relative to the visual axis.

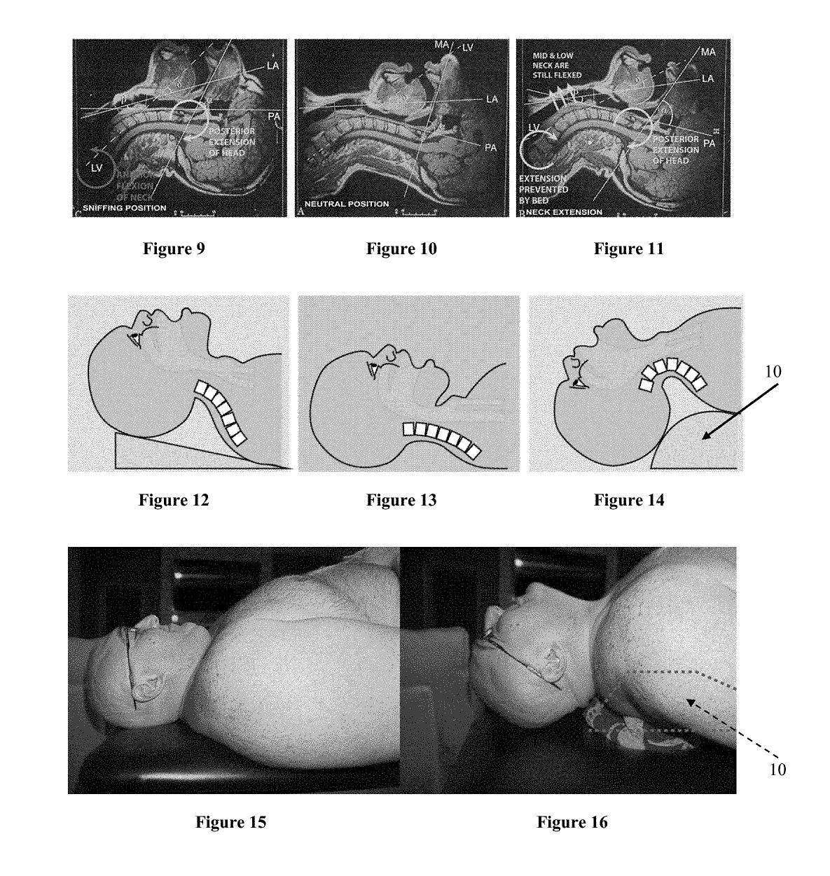

Furthermore, in the presence of the obstructing surface of a bed beneath the patient's head and neck, the real benefit of flexion in the sniffing position is to enable upper cervical extension along with the extension of the head. It is not anatomically possible to achieve significant extension at the occipitoatlantal joint without also inducing extension of the upper neck. Thus, it is extension (not flexion) of the neck that enables optimal alignment. But extension of the neck is hindered by the surface of the bed. By flexing the lower cervical spine in the sniffing position, some degree of upper cervical extension is actually enabled. It is this extension that is the key element of the sniffing position, as shown in FIGS. 9, 10 and 11.

### VIII. THE NEW POSITION: A NEW MODEL OF AIRWAY VISUALIZATION BASED ON A NEW PRODUCT AND IMPROVED USE OF MANIPULATED POSITION

In many cases, particularly in cases of morbid obesity, true extension of the entire cervical spine will provide better visualization of the vocal cords for endotracheal intubation, as shown in FIG. 12. Before the sniffing position became established dogma, many early authors described positions of extension in the process of laryngoscopy. Some of them described the head as “hanging down” in the neutral position, as shown in FIG. 13. Although the hospital bed prevents full cervical extension, a simple lift of the upper back can provide the space needed to achieve an extended position.

By providing elevation of the upper thoracic region, full extension of the entire cervical spine will provide better visualization than previously demonstrated in other positions. This new position will be referred to as the “BUMP” position as shown in FIG. 14. FIGS. 15 and 16 further demonstrate an obese patient in neutral (left) and in the “BUMP” position of full extension (right); dotted red line represents the unseen position when engaging this obese patient of an improvised “BUMP” device.

An additional benefit of using this device is that with full extension of the neck, the position for cricothyroidotomy is also optimized, should standard endotracheal intubation fail. This device is of particular benefit in the intubation of obese patients, providing good extension and adequate visualization without the need for excessive strength to manipulate the patient's head and neck.

The same position also facilitates placement of central venous catheters and other lines in the internal jugular or subclavian veins in obese or kyphotic patients. This is accomplished because in lifting the upper back, gravity causes the head and shoulders to drop back for better exposure of the neck and chest.

### IX. THE NEW AIRWAY POSITIONING DEVICE

The device of the present invention has a platform elevated by a gentle upward incline on one end and a steep drop off on the opposite end. The gradual incline allows it to be placed under the patient's back quickly and easily, without injury to the patient. The elevated plateau provides space above the bed to enable full extension of the cervical spine. Unlike other devices designed to achieve the “sniffing” or “ramped” positions, this device is different in that it does not promote cervical flexion at any level.

This device is also unique in that the plateau declines laterally on both sides in order to allow the patient's shoulders to fall backwards. This enables better positioning, especially if cricothyroidotomy is necessary. Allowing the shoulders to drop back also provides better exposure of the neck and chest, facilitating central venous catheterization of the subclavian and internal jugular veins which is often difficult in the neutral position, particularly in obese patients. The device will also have a handle at the steep end, not pictured below.

The product can be injection molded using high-density polyethylene plastic, approximately 5 mm thick. This will make the device strong, lightweight, portable, and durable. Portability and durability, although an advantage in the emergency room setting, is even more significant in the first responder environment out in the field. As the height of the plateau will need to be greater for larger patients, It will be available in several stackable sizes. The ideal dimensions and the angles of the various inclines have not been fully determined yet and will be a work in progress. The device could also be padded, and optionally adjustable in height. A variation may also fashion the device as a portable emergency response case of kit within which other airway supplies may be stored and carried.

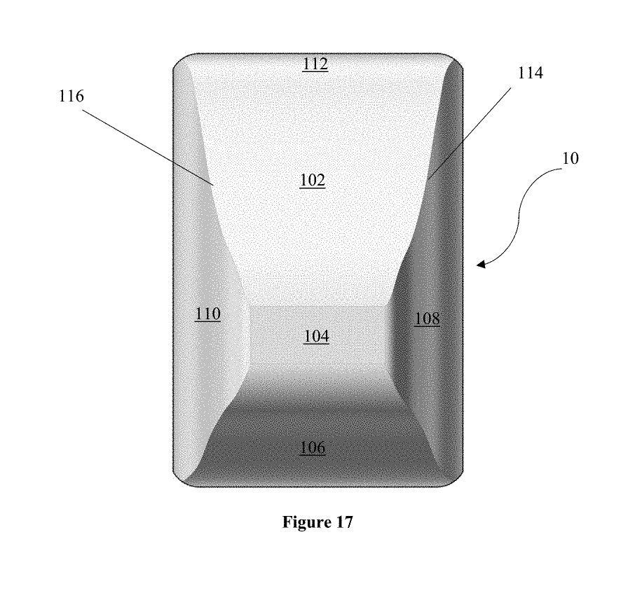

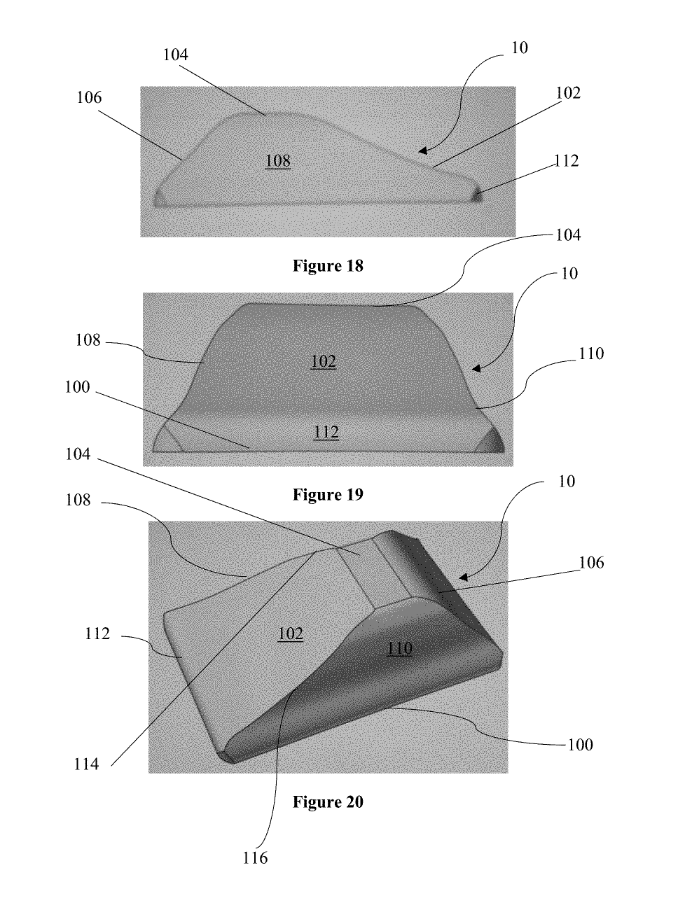

As shown in FIGS. 17, 18, 19 and 20, the device 10 has a main block 12 with a flat or substantially flat bottom surface 100, a primary ramped surface 102, a plateau support surface 104, a rear surface 106, and side supporting surfaces 108 and 110. The surfaces work together to provide the needed positioning as described above. The relationship of the surfaces is such that the symmetry allows for placement of the device 10 between the shoulder blades of a person so that the neck can fall rearwardly and the shoulders can fall to the sides, and the neck and head falling downwardly, particularly in morbidly obese patients, although the device can be utilized with all patients. The force of gravity is normally enough to hold the patient in place on the device 10. For smaller people and children, a smaller size may be optimal. Similarly, for larger people, there may be a larger size available.

The exact specifications can vary, but proportions exist wherein the primary ramped surface rises at an angle of approximately 20 degrees with the bottom surface and extending from a front fall off surface 112 to the plateau support surface 104. The rear surface ramps down at approximate 50 degrees with the bottom surface. The side surfaces ramp down from the plateau support surface 104 at approximately 60 degrees with the bottom surface. The angle measurements are taken and observed from a vertex intersecting with a line within the bottom surface and a line within the respective rear surface, side surface, fall off surface, or primary ramped surface respectively.

The bottom surface 100 has length to width proportions of approximately a length 1.5 times its width. It is a solid surface is the described embodiment, but may have other alternate configurations. The height of the plateau support surface 104 from the bottom of the device is approximately ⅓ the length of the device. Typically it would be 4 inches high on a length of approximately 12 inches long. An alternate version may be 6 inches high on a length of approximately 18 inches long. Various other versions (and heights and lengths) are also included in the invention. No sharp edges exist on the embodiment as shown, with various levels of rounded edges 114 and 116 as shown.

The plateau support surface 104 can be a flat surface or preferably be a surface with a slight angulation of approximately 3 to 5 degrees from a line parallel and included in the bottom surface 100 of the device where it ramps in the same direction as the primary ramped surface 102.

Various embodiments and variations have been discussed in the foregoing description. However, the embodiments discussed herein are not intended to be exhaustive or limit the invention to any particular form. The terminology which has been used is intended to be in the nature of words of description rather than of limitation. Many modifications and variations are possible in light of and within the above teachings and the invention may be practiced otherwise than as specifically described.

What is claimed is:

1. A product for positioning a human body comprising
A block having:
A substantially flat bottom surface;
A primary ramped surface;
A plateau support surface;
A rear surface; and
At least one side supporting surface.

2. A product in accordance with claim 1, further comprising the primary ramped surface rising and extending from a front fall off surface to the plateau support surface.

3. A product in accordance with claim 2, wherein the primary ramped surface rises at an angle of approximately 20 degrees with the bottom surface.

4. A product in accordance with claim 1, further comprising the rear surface ramps down at approximate 50 degrees with the bottom surface. The side surfaces ramp down from the plateau support surface at approximately 60 degrees with a line included in the plane of the bottom surface.

5. A product in accordance with claim 1, further comprising the at least one side surface ramps down from the plateau support surface at an angle of approximately 60 degrees with a line included in the plane of the bottom surface.

6. A product in accordance with claim 1, further comprising wherein the relationship of the surfaces is such that the symmetry allows for placement of the block between the shoulder blades of a person so that the neck can fall rearwardly and the shoulders can fall to the sides, and the neck and head falling downwardly.

7. A product in accordance with claim 1, further comprising the bottom surface having a length to width proportion of approximately a length 1.5 times its width.

8. A product in accordance with claim 1, further comprising the height of said plateau support surface from the bottom of the device is approximately ⅓ the length of the device.

9. A method to position a human body using a block having various surfaces, wherein the relationship of the surfaces is such that the symmetry allows for placement of the device between the shoulder blades of a person so that the neck can fall rearwardly and the shoulders can fall to the sides, and the neck and head falling downwardly.

10. A system to position a human body wherein the relationship of the surfaces is such that the symmetry allows for placement of a block between the shoulder blades of a person so that the neck can fall rearwardly and the shoulders can fall to the sides, with the neck and head falling downwardly by the force of gravity alone to expose the throat area of a patient to be intubated.
